(19) United States
(12) Patent Application Publication
(10) Pub. No.: US 2008/0039698 A1
(43) Pub. Date: Feb. 14, 2008

(54) Cognitive Processing

(76) Inventor: Peter Geoffrey Burton, Sydney (AU)

Correspondence Address: SUGHRUE MION, PLLC, 2100 PENNSYLVANIA AVENUE, N.W., SUITE 800, WASHINGTON, DC, 20037, US

(21) Appl. No.: 10/568,757

(22) PCT Filed: Aug. 20, 2004
(86) PCT No.: PCT/AU04/01120

§ 371 Date: Jan. 3, 2007

(30) Foreign Application Priority Data
Aug. 21, 2003 (AU) 2003904489

(51) Int. Cl. A61B 5/16

(52) U.S. Cl. 600/300; 128/898

(57) ABSTRACT

Methods for the determination of cognitive processing of an individual or groups of individuals are described. Deviation from a control range of cognitive processing is determined with reference to a standardized task. Methods comprise measurement of time to complete the standardized task in a group of control subjects, measurement of temporal eyeblink occurrence during performance of the task by the control subjects, and calculation of a control range for the control subjects, the control range being calculated from at least the temporal eyeblink occurrence during a common phase within the standardized task for the control subject. Deviation from the control range indicates the test subject has altered cognitive processing relative to the control subjects.

### FIELD OF THE INVENTION

The present invention relates broadly to methods and devices for measuring cognitive processing (including cognitive processing ability, performance, aptitude or capability) in a subject, and more specifically, to methods and devices using eyeblink parameters for measuring the cognitive processing of the subject.

### BACKGROUND OF THE INVENTION

An individual's cognitive performance is a parameter that is difficult to measure with any degree of objectivity. It can, for example, be altered transiently, such as by way of fatigue or stress, which are two conditions or states widely considered to compromise cognitive performance relative to the underlying level of ability when alert and relaxed, respectively. The underlying level of cognitive ability of an individual is itself compromised either transiently or more systematically as a result of trauma (such as Post-Traumatic Stress Disorder, PTSD) or disease (such as dementia associated with Alzheimer's Disease, AD), respectively.

Defining objective criteria medically relating to cognitive performance, such as in depression, has been hindered by the subjective nature of self-reports which are often the sole direct evidence for (a) the particular states of an individual from time to time, (b) the longitudinal evolution of state(s) in an individual over time, and (c) the comparison of an individual with supposed peers. Traditionally the scoring of a subject on various types of aptitude tests has been the basis of assessment of cognitive ability, but these tests reflect not only intrinsic cognitive ability but also prior cultural, economic and educational circumstances of the individual, factors which are themselves predictive of future performance so confounding the tests results in respect of intrinsic ability at a given time.

In order to assess intrinsic cognitive ability, psycho-medical science would like access to indicators of underlying brain-processing capability. Unfortunately, no theory is available to guide the development of such indicators. Empirical brain activity measures (electroencephalograph or EEG analysis) and structural brain imaging (positron emission tomography or PET, functional magnetic resonance imaging or fMRI) straddle the time domain of likely strings of coherent brain activity (the former being too short with measurements focussed upon the second following stimulus; the latter subject to repetitive statistical measurement protocols of simple first order tasks, but with at least ten of seconds, possibly tens of minutes, of data collection time). Cognitive activity tends to occur as coherent forms of processing over burst periods of seconds to tens of seconds, out of ‘range’ of EEG (which is too short term, focussing on Event-related Potentials, ERP up 1000 ms), out of match with the multiple repeat statistical sampling rates required for fMRI, and too fast for PET.

Current understanding of blinking of an individual is that blinks are merely parameters of eye-physiology, and as general arousal indicators. It is known, for example, that the subjective experience of perceiving visual stimuli is accompanied by objective neuronal activity patterns such as sustained activity in primary visual area (V1), amplification of perceptual processing, correlation across distant regions, joint parietal, frontal, and cingulate activation.

However, the value of the eyeblink in assessment of cognitive fitness has not been appreciated. For example, in the EEG literature, eyeblinking is regarded for its undesirable noise value, that is, it represents artifact (Jung et al, Removal of electroencephalographic artifacts by blind source separation, Psychophysiology 37 (2000) 163-178) which distorts the power spectrum of the classic EEG bands. This occurs particularly when proximity with respect to the frontal lobes, where the change in capacitance of the eyeball socket during a blink creates a changing electric field that registers within EEG sensors. Hence, no value has been afforded to detailed eyeblink information in standard medical testing procedures. Prior publications merely refer to ‘average’ blink rates as being sensitive to cognitive load and arousal/fatigue dimensions (NASA TP-2001-211018).

Accordingly, the present invention seeks to provide a method of blink pattern analysis in relation to cognitive tasks in a natural or office setting, these methods being designed to objectively characterise brain activity over coherent strings (or larger chains of these) of focussed cognitive activity. These methods seek to provide a means of discriminating the cognitive performance of an individual across internal states and external task settings, longitudinally across time for those same states or settings, and between individuals.

### SUMMARY OF THE INVENTION

In its broadest form, the present invention relates to a method for creating a control range suitable for detecting cognitive processing of at least one test subject using a standardised task, the method comprising:
measurement of time to complete the standardised task in a group of control subjects; and
measurement of temporal eyeblink occurrence during performance of the task by the control subjects; and
calculation of a control range for the control subjects, the control range being calculated from at least the temporal eyeblink occurrence during a common phase within the standardised task for the control subjects;
   wherein deviation from the control range indicates the test subject has altered cognitive processing relative to the control subjects.

Preferably, temporal eyeblink occurrence is used to derive the gap or time elapsed between blinks over a plurality of adjacent blink events.

In a particularly preferred form, the standardised task is a structured task, where the common phase is selected from:
a first orientation phase, occurring at commencement of the task;
a second or intermediate phase showing the test subject's task progress; and
a common task completion period (CTCP) of the task.

Preferably, the structured task is chosen to deemphasise the control subject's mental processing during the orientation phase and/or intermediate phase of the task by incorporation of the orientation phase and/or intermediate phase into later phases of the task.

Even more preferably, the common phase of the standardised task is the completion phase of the task.

In a further preferred form, measurement of at least one of eyeblink characteristic of eyeblink power B is also performed, the eyeblink characteristic being selected from eyeblink duration and eyeblink amplitude, the eyeblink characteristics occurring during performance of the standardised task.

In another preferred form, the common phase of control subject's performance in the common phase of the task is analysed by plotting quantitative gap time elapsed between each blink against time of the task.

Preferably, measurement of at least one eyeblink characteristic of eyeblink power B is also plotted against time of the common phase of the task, wherein B is selected from eyeblink duration, eyeblink amplitude, temporal gap between eyeblink occurrence or any derivative thereof.

In a particularly preferred form, individual blinks are smoothed and weighted by clustering into peaks occurring over the common phase within the task.

In yet another preferred form, a smoothing function F is used to compare blink clusters on a two-dimensional scale of blink density against time for the common phase of the task, and wherein number of blinks per cluster is estimated by the peak area above an estimate of the background blink rate (BB), with the converse measure being the proportion of time that blink density does not exceed the baseline of the background blink rate of the blink density.

Preferably, individual blinks are weighted by assigning a weighting using variables selected from unit weighting, absolute amplitude value (a), absolute duration value (d), absolute gap value (G) or a weighting derivative thereof.

In another form, the method further includes interpretation of the control subjects' performances in the standardised task using additional parameters for each control subject, including at least one intensive parameter calculated from the common phase of the task and at least one extensive parameter of blink patterns for the entire task, wherein the intensive parameter is selected from the group comprising:
percent of blinking time in the CTCP, when blink density is above a baseline blink density (I1);
mean cluster size (integral of height of density divided by number of peaks in last CTCP, representative of blinks clustered at each concentration release, whereby blink clusters are determined by measuring the area of each cluster peak above the local baseline) (I2);
average baseline blink density in last CTCP (derived from the opening function by calculation of the incidence of minimum followed by a maximum in the relative density function within a threshold of 15 seconds or 10%-20% of the CTCP) (I3); and
average blink rate for the Task (calculated by total number of blinks over the Task on total time taken for the entire Task) (I4),
   wherein I1-I3 are derived using a smoothed blink density function F for each blink, and wherein the extensive parameter is selected from the group comprising:
total duration of Task (E1 or T);
total number of blinks (E2 or N);
number of clusters during entire Task time (peaks in blink density) (E3); and
indirect measure of number of clusters/attempts at stages in task (E4), using a formula of attempts (A)=T̂2/sum(gap̂2);
   wherein variation in one or more intensive and one or more extensive parameters calculated for the control subjects provides data for the control range for the one or more intensive or extensive parameters.

Preferably, the smoothing function F is a normal Gaussian function.

In one preferred form, the control group consists of subjects with normal cognitive function.

In a further preferred form, the control group consists of subjects with compromised cognitive function, such as compromised cognitive function associated with a disorder such as ADD, ADHD, dyslexia, dementia, schizophrenia, depression, learning disorders, sleep disorder, stress disorder, personality disorders, borderline personality disorders, or cognitive function impaired or enhanced by alcohol or drug ingestion.

In yet another preferred form, the control group consists of subjects with enhanced cognitive function.

In another form, the invention relates to a method for detecting cognitive processing of at least one test subject using at least one standardised task, the method comprising:
measurement of the subject's time to complete the standardised task; and
measurement of temporal eyeblink occurrence; and
comparison of the temporal eyeblink occurrence or a value derived therefrom, during a common phase within the standardised task, with a control range, the control range calculated from at least the temporal eyeblink occurrence during the common phase within the standardised task for a control group;
   wherein deviation from the control range indicates the test subject has altered cognitive processing relative to the control group.

Preferably, the control range is calculated according to the methods of the invention.

Even more preferably, the test subject's cognitive processing is compared with a control range of one or more intensive and a control range of one or more extensive values.

In a particularly preferred form, the intensive value is I4, and the extensive value is E3.

Preferably, the test subject's cognitive processing is tested to detect cognitive disorder selected from the group consisting of ADD, ADHD, dyslexia, dementia, schizophrenia, depression, learning disorders, post-traumatic stress disorder, sleep disorder, personality disorders, borderline personality disorders, or cognitive function impaired or enhanced by alcohol or drug ingestion.

In a further form, the present invention relates to a method for creating a control range for detecting cognitive processing of at least one test subject using a standardised task, the method comprising:
measurement of time to complete the standardised task in members of a group of control subjects and measurement of temporal eyeblink occurrence and total eyeblink number N, for the control subjects; and
sorting control subjects from lowest to longest time to complete the task and analysing each control subject having a similar task completion time with respect to their N value;
creation of at least one control range of N values according to the time to complete the task,
   wherein deviation from the control range indicates a test subject having altered cognitive processing relative to the control subjects having a similar task completion time.

Preferably, the standardised task is a structured task, where the common phase is selected from:
a first orientation phase, occurring at commencement of the task;
a second or intermediate phase showing the test subject's task progress; and
a common task completion period (CTCP) of the task.

Even more preferably, the structured task is chosen to deemphasise the test subject's mental processing during the orientation phase and/or intermediate phase of the task by incorporation of the orientation phase and/or intermediate phase into later phases of the task.

Preferably, the common phase of the task is the common task completion period (CTCP) of the task.

In a preferred form, the control subjects' times to complete the task are firstly sorted by adjusting blink patterns to align the common completion phase of the task, prior to comparing of control cohort members with similar task completion times with respect to their N values during the common phase of the task.

Preferably, measurement of at least one of eyeblink characteristic of eyeblink power B is also performed, the eyeblink characteristic selected from eyeblink duration and eyeblink amplitude, wherein the eyeblink characteristic occurs during performance of the standardised task.

Even more preferably, the method further includes interpretation of the control subjects' performances in the standardised task by linear regression using additional parameters for each control subject, the additional parameters including at least one intensive parameter calculated from the common phase of the task, and at least one extensive parameter calculated over the entire task, the intensive parameter being selected from the group comprising:
percent of blinking time in last or common task completion period (CTCP), when blink density is above a baseline blink density (I1);
mean cluster size (integral of height of density divided by number of peaks in last CTCP, representative of blinks clustered at each concentration release, whereby blink clusters are determined by measuring the area of each cluster peak above the local baseline) (I2);
average baseline blink density in last CTCP (estimated from the opening function by calculation of the incidence of minimum followed by a maximum in the relative density function within a threshold of 15 seconds or 10%-20% of the CTCP) (I3); and
average blink rate for the task (calculated by total number of blinks over the task on total time taken for the entire task) (I4),
   wherein I1-I3 are derived using a smoothed blink density function F for each blink, and wherein the extensive parameter is selected from the group consisting of:
total duration of task (E1 or T);
total number of blinks (E2 or N);
number of clusters during entire task time (peaks in blink density) (E3); and
indirect measure of number of clusters/attempts at stages in task (E4), using a formula of attempts (A)=T̂2/sum(gap̂2);
   further wherein the linear regression is used to establish a linear combination of at least one of I1 to I4 and at least one of E1 to E4 which best represents the structure of the blinking patterns in the control group, wherein variation in one or more intensive or extensive parameters calculated for the control subjects provides data for the control range for the parameter derived by linear regression.

Preferably, the smoothing function F is a normal Gaussian function.

In a particularly preferred form, the structure of the blinking patterns in the control group is classified by ranking subjects according to three ranges to which are assigned values between 1 and 3, wherein 1 represents highly structured or sparse blinking, and 3 represents struggling eyeblinking with an eyeblink pattern of low structure or dense blinking, and 2 represents an intermediate structure of eyeblinking, for similar task completion times or for the common phase of a task.

In a preferred form, the control group consists of subjects with normal cognitive function.

In a further preferred form, the control group consists of subjects with enhanced cognitive function.

In yet another preferred form, the control group consists of subjects with compromised cognitive function, such as compromised cognitive function associated with a disorder selected from the group consisting of ADD, ADHD, dyslexia, dementia, schizophrenia, depression, learning disorders, post-traumatic stress disorder, sleep disorder, personality disorders, borderline personality disorders, or cognitive function impaired or enhanced by alcohol or drug ingestion.

In yet another form, the present invention relates to a method for detecting cognitive processing of at least one test subject using at least one standardised task, the method comprising:
measurement of time to complete the standardised task and measurement of temporal eyeblink occurrence and total eyeblink number N, for the test subject; and
analysing the test subject with respect to their N value;
comparison of the test subject's time to complete the task and N value with a control range for time to complete the task and N values,
   wherein deviation from the control range indicates a test subject having altered cognitive processing relative to the control range.

Preferably, the control range is calculated according to the methods of the invention.

Even more preferably, the test subject's cognitive processing is compared with a control range selected from intensive and extensive values.

Preferably, wherein the intensive value is I4, and the extensive value is E3.

In a particularly preferred form, the intensive value is derived from I4 and E3.

In one form, a set of ranking values for each subject in a control group is used to calculate an Objective Structure Index by linear regression from a set of intensive and extensive parameters calculated for each subject, where the resulting linearised Objective Structure Index value for the test subject is plotted as an intensive variable relative to the range for the control group, and further this plot in one dimension is displayed against the corresponding plot in a second dimension representing an extensive variable, wherein the extensive variable is T or N or a function of T or N.

Preferably, the test subject's cognitive processing is tested to detect a cognitive disorder selected from the group consisting of ADD, ADHD, dyslexia, dementia, schizophrenia, depression, learning disorders, post-traumatic stress disorder, sleep disorder, personality disorders, borderline personality disorders, or cognitive function impaired or enhanced by alcohol or drug ingestion.

In yet another form, the present invention relates to a device suitable for recording temporal occurrence of eyeblinks during a time taken for a subject to complete a standardised task, according to the methods of the invention.

In a further form, the invention relates to a device suitable for recording temporal occurrence of eyeblinks during a time taken for a subject to complete a standardised task, and eyeblink characteristics of eyeblink duration and eyeblink amplitude, according to any of the methods of the invention.

In another form, the invention relates to a device suitable for displaying temporal occurrence of eyeblink during a time taken for a subject to complete a standardised task, according to any of the methods of the invention.

In a further form, the invention relates to a device suitable for displaying temporal occurrence of eyeblinks during a time taken for a subject to complete a standardised task, and eyeblink characteristics of eyeblink duration and eyeblink amplitude, according to any of the methods of the invention.

In another form, the present invention relates to the use of a device suitable for recording temporal occurrence of eyeblinks during a time taken for a subject to complete a standardised task, for performing the any of the methods of the invention.

In yet another form, the invention relates to the use of a device suitable for recording temporal occurrence of eyeblinks during a time taken for a subject to complete a standardised task, and eyeblink characteristics of eyeblink duration and eyeblink amplitude, for performing any of the methods of the invention.

In a further form, the invention relates to the use of a device suitable for displaying temporal occurrence of eyeblinks during a time taken for a subject to complete a standardised task, for performing any of the methods of the invention.

In another form, the invention relates to the use of a device suitable for displaying temporal occurrence of eyeblinks during a time taken for a subject to complete a standardised task, and eyeblink characteristics of eyeblink duration and eyeblink amplitude, for performing any of the methods of the invention.

In yet another form, the present invention relates to a method of computational analysis for analysing eyeblink data, in order to create a control range for cognitive processing and/or detect the cognitive processing of a test subject, according to any one of the methods of the invention.

In another aspect this invention is concerned with assessing cognitive processing by the pattern of eyeblinks in a standardised task. Accordingly, the invention in this aspect extends to a method of assessing cognitive processing of a subject by analysis of eyeblink pattern in a standardised task.

### BRIEF DESCRIPTION OF THE DRAWINGS

FIG. 1: Schematic representation of a maze navigation task as a standardised task example.

FIG. 2: Schematic representation showing graphs displaying interval between blinks for the concluding phase of a standardised task.

FIG. 3: Schematic representation showing graphs of five sample control subjects displayed as two-dimensional displays showing both blink incidence and blink power (B=a*d) for each blink during completion phase of a standardised task.

FIG. 4: Schematic representation showing blink data plotted against time on the left for five control subjects, and smoothing of blink data (replacing each blink with a smoothing function F) into a ‘density’ D of blinks to show the clustering of blinks into peaks during the CTCP phases of a task (last 90 seconds shown as common across subjects selected).

FIG. 5: Schematic representation showing graphical comparison of unit weighting (bw=3 seconds, left) and a*d/G weighting (bw=1.5 seconds, right) showing peak count in density functions over last 90 seconds and baseline periods of relative unblinking.

FIG. 6: Graphical representations showing number of blinks for subjects during a common completion phase of a standardised task.

FIG. 7: Schematic representation showing graphical ordering control subjects and ADHD subjects by completion Time Ti.

FIG. 8: Schematic representation showing joint distributions of Control subjects over task Completion Time and Objective Structure Index.

### DETAILED DESCRIPTION

The present invention is predicated on the realisation that sophisticated information regarding cognition can be obtained by analysis of blink patterns in a controlled setting, such as by the information revealed by blink interval correlation and/or blink duration. This valuable information can be utilised to detect local cognitive effects. In particular, the invention is based on the discovery that blinking can be used as a measure of cognitive state of an individual at the time of testing. This state may vary from time to time and from individual to individual, but the richness of blinking data is such that this state can be compared for systematic changes across time which reflect changes in internal processing, since blinks are for the most part subconscious events.

Accordingly, blinking, and in particular blink patterns and clustering, may therefore used to reveal the actual timecourse of real-time processing active in the brain in undertaking a task.

### Eyeblinks Generally

Blinks occur for various reasons, but the central point of the present invention is that some blinks occur at times when central brain neurotransmitter activity leads to a gating event. Gating is a term that has been used to refer to neurological activity surrounding the thalamus and is thought to be associated with the appraisal (sometimes known as binding) of data from the different senses in perception. The eyeblink becomes associated with gating activity as a learned phenomenon, to minimise the visual input loss of blinking through preferably timing blinks to occur within the dead-time of the gating event. However, rather than reflecting activity only in the visual circuits of the brain, eyeblinks come to reflect aspects of whole-of-task processing in the brain. Our use of the term gating refers to all process pauses whether they be related to perceptual input, central processing or modulation of action output. Thus the general term used to reflect the break up of brain processing into sequential subtasks is referred to here as “gating”.

Gating associated with thought (and neither active perception nor action output) is revealed in the eyeblink residue activity one senses which occurs to reflect process punctuation even while the eyes are held closed.

Therefore blink analysis reflects not so much what the brain is processing from time to time, but how it processes the task in time. The notion that conscious task processing occurs in discrete subunits of content has been understood in some fields for some time, but the notion of how this process is broken up in real time is new to the present invention.

### Types of Eyeblinks

Certain types of blinks are triggered by an external, identifiable stimulus (eg startling of a subject, an airpuff delivered to the eyeball, and the like). These blinks tend to be involuntary.

As referred to herein, the terms “stimulated blink” or “exogenous blink”, or plural terms thereof, mean eyeblinks elicited by an identifiable, external stimulus or basic physiological requirement associated with the anatomical requirements of the eye, such as the above-mentioned airpuff to the eyeball, the lubrication/wetting of the surface of the eye, or reflex blinking to a stimulus. Such stimulated or exogenous blinks exclude those that are associated with cognitive processing.

Another class of blinks is voluntary or conscious blinks, such as occur when the attention is directed from one field or context of attention to another, or in deciding to move from one task to another. These may be minimised by investigating blinks on a given task which holds the attention of the subject sufficiently or, preferably, programmed in to the definition of a Structured task (see below).

The phrases “eyeblink associated with cognitive processing” or “cognitive processing eyeblinks” or “endogenous eyeblinks” (Stern, Walrath & Goldstein, ‘Endogenous. Eyeblinks’ Psychophysiology, 21 (1984) 22-33). This publication differentiates voluntary, startle & reflex blinks from central or endogenous blinks, and describes an eyeblink event occurring in the absence of any identifiable, eliciting stimulus, but which occurs while processing a single task, and are typically involuntary. An example is the blink that tends to occur at the end of a series of saccades towards a fixation point in vision.

Cognitive processing eyeblinks in a subject can be indicative of stages within a subject's performance of a task, namely, a) registration of novel information, b) high-level cognitive processing, c) task mastery, and d) fatigue or boredom. When a subject performs a standardised task, the aforementioned stages of task processing have been found by the inventor of the present application to be characterised by different and distinctive blink patterns. Major blinks are longer and deeper blinks (full eye closure), while a blink type classified as a miniblink represents a shorter and shallower blink (partial eye closure). No isolated blink on its own, nor any average measure over many blinks (such as a blink rate) on its own, is informative of the brain's cognitive processing, but the present inventor has found that a pattern of incidence of major blinks and miniblinks during the passage of a task does reveal important and useful aspects of the brain's cognitive processing.

Major blinks tend to delimit periods of coherent central task processing, whereas miniblinks tend to occur within bursts of coherent task processing. Conversely, miniblinks tend to occur within periods of sustained attention, while major, deeper blinks tend to occur between two periods of sustained attention.

As referred to herein, the term “miniblink” thus refers to a type of blink exhibiting identifiable physiological characteristics, including shorter duration and shallower depth, relative to a major blink, and being an indicator of a subject's internal processing in performing one or more phases of a standardised task.

Major blinks tend to occur at points of change of attention of a subject, with relatively large blink amplitude and duration values being shown, whereas miniblinks tend to occur within a period of sustained attention with relatively small amplitude and duration values. Thus, major blinks tends to occur between phases of mental processing of the task, while miniblinks tend to occur within the given phase of a task. Accordingly, by characterisation of blink type there is provided a means of segregating the task record into periods of sustained mental effort. These periods tend to be delimited by major blinks or blink clusters, which occur in quick succession as an endpoint in a phase of a task is being approached or as a flutter of blinks as concentration is released after its achievement. Thus, the blink record during the test is a means of interpreting phases of mental processing used by a subject to complete the standardised task.

Preferably, these phases of mental processing can be emphasised for the purpose of the comparison of different subjects, by the choice of a standardised task which has phases that incorporate the passage of earlier phases of the task into the execution of subsequent phases of the task (referred to herein as a “structured task”) where memory is required to be used to progress the task.

It is preferable that for the methods of the present invention, a structured task is performed in an environment that is selected and/or designed to reduce exogenous blinking in a subject, that is, blinks that are elicited by an identifiable stimulus, mentioned above. Broadly, this involves minimising distractions from the task performance, as would be typical in laboratory or study settings.

### Measurement of Eyeblinks

Measurement of detailed blink characteristics during a task shows temporal structure reflecting cognitive processing, that is, how the brain breaks down completion of a task into subtasks.

The term “cognitive processing” as used herein thus refers to an intrinsic capacity of a subject to perform a certain activity or task, wherein the activity or task requires the brain to fulfil a series of requirements, such that the cognitive processing is indicative of the subject's neurochemical makeup and neurochemical gating.

This neurochemical gating phenomenon is thought of as being similar to the role played by punctuation marks in breaking up written text into meaningful phrases. The serial (sub-) tasks to which attention is addressed in conscious processing by the brain is thus regarded as a sequence of ‘episodes’, each of which is regarded as a subunit of coherent attention. This attention may be directed towards the external world (perceptual processing or action on it), or be internal to the mind (i.e. thought).

Each ‘episode’ occurs within the delimiters of two gating signals, and so refers to a single focussed burst of coherent processing. Within an episode, input processes, central processes and output processes will typically occur with a certain degree of overlap.

The present invention recognises that coherent cognitive processing occurs in short bursts delimited by major attention changes and invariably blinks, just as the inspiration breath during speech production forms a natural punctuation between coherent bursts of utterance. Averaging across changes of attention will wash out any data reflective of coherent bursts of cognitive processing on input. So it is the short-term order, and the local-in-time cross-correlation between blink interval and ensuing blink duration, which must be tracked to discern regularities associated with internal cognitive processing modes. This tracking of eyeblinks in time, and local correlation in time of blink interval, represents a first dimension of analysis required for the methods of the present invention.

In one embodiment of the present invention, the blinks are also accurately tracked in time to characterise both their nature (depth/duration) and their relative local incidence (interval, nearest neighbour intervals, etc.), as independent measures so that joint measures taken from these two dimensions can discriminate epochs of cognitive processing (say 2-5 s induration).

The methods of the present invention collect information in the time domain, not the frequency domain (its inverse) characteristic of EEG reports. Direct measurement of eyeblinks preferably occurs via an electro-oculographic device (which records an EOG voltage representing the potential difference between the cornea and the retina or fundus). The EOG voltage is created by movement of the eyelid, creating a positive charge in the cornea, with respect to the fundus. Through EOG measurements, the position of the eyelid during a blink can be recorded. However, EMG (electromyographs) can also be used for the methods of the present invention, as these devices can record electrical activity from the muscles that initiate eyelid closure (eg the orbicularis oculi). Even simpler devices may also be employed, such as direct attachment of a string to the eyelid, and using a potentiometer to measure closing and opening of the eye. Other techniques, such as photo-electric recordal of scleral reflection or other reflections from the eye, including film, video and CCD image records which may be analysed offline, may be employed for the methods of the present invention, as a means of detecting the timecourse and shape of each eyeblink occurrence.

Measurement of the blink time parameters simplifies data collection apparatus so that measurements may be taken in a much wider group of settings that is possible for (electroencephalogram) EEG measurements.

Furthermore, the invention recognises that merely measuring the blink depth parameter (a) (oreyeblink depth closure, perclos, percentage of iris covered by the eyelid) only provides information related to fatigue or alertness, the activation of the experimental subject. However, blink depth or amplitude does provide valuable information about each eyeblink, when combined with the duration (d) of the eyeblink. The combination of a large amplitude and duration for an eyeblink signifies a major blink which may be involuntary between subtask phases of a task or voluntary to refresh processing resources ahead of a new phase of a task. One may liken the latter to a paragraph break in written text which signifies a major change of attention, compared to smaller pauses characteristic of fullstops or commas within a paragraph of such text which signify process points within the one field of attention.

When two dimensions (a and d) to characterise each blink of a data set are collected for a subject, these may be combined to provide a general measure (τ) of the blink significance, such as the area under a blink trace (τ approximated by the “blink power” parameter B=a* d) to assist in placing the blink on the continuum from miniblink through major blink to extended voluntary blink.

The timecourse of blinks on a task may be discerned more readily from the fact that eyeblinks occur with a minimum refractory period between adjacent blinks which itself may be informative of cognitive processing. Thus the timecourse of occurrence of each of a series of adjacent blinks may be used together with the time of occurrence and therefore the interval between individual blinks.

Accordingly, the term “Gi” is used herein to denote the interval or gap from the previous blink to blink i, where i refers to an individual blink.

Preferably, the correlation between two independent parameters, one reflecting the previous blink interval or time gap Gᵢ=tᵢ−tᵢ₋₁, and one reflecting the individual blink significance Bᵢ=aᵢ*dᵢ, is examined, and higher-order correlation data examined for cluster analysis within temporal blink patterns and their amplitudes.

The term “higher-order correlation data” as used herein refers to mathematical functions applied to calculate, smooth and/or weight individual blink incidence data, to visualise, count and compare clusters of blinks. The smoothing of blink incidence by replacing each event with a normalised Gaussian function F having a standard deviation commensurate with the minimum blink interval is a preferred means to automatically detecting blink clusters.

According to the present invention, relatively isolated individual blinks, neighbouring pairs of similar blinks, neighbouring triples and even larger clusters are contributors to valuable diagnosis and discrimination of cognitive processing. This becomes apparent when individuals are compared for their performance on similar phases of a particular structured task. Even when comparing two subjects with task completion times that are relatively close to each other, we can observe large differences in individual processing characteristics between individuals through the sparseness or clustering of blinks in their eyeblink records during the task.

The two parameters of gap time and blink significance also allow the data collected to be displayed in a manner which is highly informative of the actual timecourse of cognitive processing in relation to specific aspects of the standardised tasks, discussed in more detail below (designed to assess cognitive processing using iterative and cognitive requirements), including the period in which instructions are being given to the subject. This allows serial data to be subdivided into batches which pertain to certain kinds of cognitive tasks, and in particular, phases within a task.

Preferably, these measurements quantify characteristics of personal cognitive processing structure and so compare performance of individuals within certain experimental settings, such as those relevant to tasks anticipated in work environments. Learning performance comparisons then enable selection of individuals most suited to the proposed task structure, and furthermore for each individual, allows the objective identification of factors which are detractors of optimal performance of an individual.

The methods and devices of the invention are proposed for use particularly as a subject-screening means, in contexts such as military, safety, industrial process and financial market settings, as well as educational settings. In particular the training rate of each individual is useful as a predictor of meeting sustained performance objectives by that individual. It will be understood that such detection methods will also be of value in more general educational or training settings to objectively assess the learning competence of an individual against his/her peers with prescribed subject matter.

The particular utility of tracking eyeblink data for individuals is that, with the exception of occasional voluntary blinks (or any other exogenous blinks), the endogenous blinks which reflect gating and subtask processing punctuation are subconscious events for the subject so they reflect the natural processing style of that individual in the experimental situation. Furthermore, the richness of the data which arises from the frequency of blink events, may be used to track the performance of an individual through differing phases of a task, across different instances of that task (to show a learning effect in the short term or possibly a performance degradation in the longer term), or to compare one individual with another or a group of supposed peers.

The methods and devices of the invention also seek to provide a systematic basis for characterising and in particular, detecting, normal and abnormal cognitive performance metrics, which are proposed to complement the diagnostic criterion of any particular neurological and psychiatric illness and disorder as currently described in DSM IV (and sequelae) (The Diagnostic and Statistical Manual of the American Psychiatric Association, version IV, 1994). Accordingly, the methods and devices of the invention also seek to provide an objective basis for the measurement of the impact of treatments (particularly pharmacological treatments, nutraceutical or dietary changes, and counselling or behavioural therapy) of that illness or disorder. Thus the device and measurement system will have major use in analysing the efficacy of drug and other treatments for psychiatric illness, including but not limited to ADHD, depression, schizophrenia, OCD, autism and Parkinson's Disease.

Additionally, the methods and devices may be used to detect psychometric parameters, such as concentration changes in a subject or other types of cognitive processing variations, which can occur in contexts that include, but are not limited to, drug or alcohol ingestion.

The various roles of the monoaminergic neurotransmitters dopamine (DA), noradrenaline (NA) and serotonin within cognitive processing show relation to certain aspects of blink activity, which thus provides the psychophysiological link between abnormal blinks and psychiatric and psycho-disorders. Blinks are phasically correlated with general monoaminergic neurotransmitter gating activity in the brain, so monoaminergic or catecholaminergic (DA, NA) disorders or drugs are expected to be a major application of this invention. An advantage of the present methods is that the measurement techniques are non-invasive to provide an objective quantification of brain activity directly related to higher brain function. Other advantages are relative simplicity, low cost and convenience of use, in environments such as domestic settings or work environments, compared to many other ways of investigating brain activity.

The present invention thus relates to passive tracking and objective methods for characterising brain activity, particularly in relation to higher cognitive activity. The measurement process for this method analyses endogenous eyeblink activity (in particular, in a preferred instance of this device, through EOG activity) into certain blink types and adjacent interblink intervals, which unit structure provides the analytical basis for tracking and clustering of blink activity into patterns representative of certain distinct kinds of brain processing activity.

The present invention characterises blink types and blink interval characteristics in such a way as to accurately reflect underlying brain processing structure. This characterisation of underlying brain processing structure provides a means of comparing individual performance under standardised test situations, so providing the basis for data collection and comparative analysis of individual brain processing performance.

Without being limited to any single mode or theory of action, the inventor of the present application proposes that the tendency of blinks tend to occur in association with significant neurotransmitter activity related to attention changes in the brain means that brain-process timing characterises episodes of attention. The pattern of occurrence in time of blinks and the magnitude of each blink together indicate how a task is being processed into ‘punctuated’ subtasks. Furthermore, it is hypothesised that miniblinks may be likened to the types of punctuation in written text, from paragraph breaks (major focus change) to in-process stops and commas (miniphrasing markers). Thus miniblinks reflect internal brain process punctuation styles.

### Preferred Standardised Tasks

Individual differences in blinking patterns are sufficiently broad that standardised tasks are the preferred format for blink analysis. Tasks designed to incorporate repetition of some phases of a task are preferred as these will slow learning effects. Such designs tend to emphasise major task breakpoints, so introducing a degree of similarity into the blink patterns of different individuals. Even so wide diversity tends to characterise the blink patterns of two individuals engaged on a similar task. These differences provide the rich data from which subtyping of individual processing styles may be identified.

### Subject Comparisons: Standardised Tasks Selected

Preferably, when individuals are compared using the methods of the invention, the individuals perform the same standardised task, and even more preferably, subjects perform more than one standardised task. As mentioned above, in a particularly preferred form, at least one of the tasks is a structured task, in that it includes both progressive and iterative (repetitive) task elements. The structured task has phases incorporating the passage of earlier phases of the task into the execution of subsequent phases of the task, so memory is required to be used to progress the task This methodology allows certain phases within the task to be analysed, including, but not limited to:
a) orientation phase of commencement of task
b) intermediate steps of progress
c) completion with common achievement for all subjects

Where the standardised task is learned progressively, and concludes with a completion or mastery phase, it is particularly useful to compare different subjects' performances by completion phase comparison, as the learning rate differences of the subjects has less of an effect on the test performance comparison using such an analytical approach.

### Individual Factors

There is a wide range of blink patterns that characterise an individual's processing response to the task demands of a given task. This is especially pronounced if the task is novel and there is a strong learning effect from repetition of the task.

There are also wide individual differences between two individuals in processing a similar task. The comparison between individuals processing the same or similar task can reveal those individual differences.

Blink patterns for an individual change as a result of learning and familiarity with a task.
Any new task takes time to register and understand, and individual approaches to comprehension make the blink punctuation of the task generally unpredictable, but the incidence of blinking in this phase is more frequent, involving relatively deep blinks.
In a structured or repetitive task, blink patterns generally reflect the natural phases of a task.
Blink incidence is stimulated by the normal saccades in attention within a task as visual field is scanned for perceptual clarity.
Blinks tend to be suppressed during periods of concentrated mental attention. As concentration is released, a flurry of blinking tends to occur.
As task familiarity increases, blink incidence diminishes, and more shallow blinking occurs.
With fatigue or boredom, lower blink incidence but deeper blinking occurs.
Blinks tend to co-occur with action output decisions (endpoints), and word or phrase endings when a subject is processing input.

Furthermore, comparison of individual subjects reveal further, underlying similarities in blinking, including:
a) tendency to blink at successful perception;
b) tendency to blink at commencement of action;
c) tendency to withhold blinking during concentrated attention;
d) tendency to release on or more blinks (flutters of blinks) at the end of a concentration period; and
e) tendency of blinks occurring to mark internal processing phases of a task (miniblinks).

### Analysis of Data from a Standardised Task

Preferably, data from a selected, standardised task is collected from a cohort of control subjects. This cohort may consist of a group of subjects who have not been diagnosed with, one or more of ADD, ADHD, dyslexia, dementia, schizophrenia, depression, learning disorders, post-traumatic stress disorder, sleep disorder, personality disorders, borderline personality disorders, or cognitive function impaired by alcohol or impaired or enhanced by drug ingestion.

Alternatively, the control group may represent a cohort of subjects who have been diagnosed with, and/or are exhibiting the signs or symptoms of, at least one of the aforementioned conditions. This type of control group is particularly preferable when greater clarity is sought in defining objective measures of subtypes of the particular condition associated with altered cognitive processing.

The term ‘subtypes’ of a condition is used herein to refer to groupings of subjects having a particular condition associated with or characterised by altered cognitive processing, these groupings being objectively recognisable from the methods of the invention by the detail available in eyeblink data, which cannot be recognised to the same degree using normal diagnostic criteria alone (e.g. DSM IV), due to the qualitative, verbal nature of the diagnostic input data provided by the subject having the condition. Neurological syndromes that are defined with some ambiguity, as collections of signs observable in a subject, are likely to fall into subtypes only with the availability of sufficient quantitative data in several relevant dimensions to resolve them, such as the methods provided by the present invention.

In a further form, the control group consists of a cohort of individuals with a higher than average intelligence quota (IQ), and/or enhanced learning ability, memory skills or other improved cognitive function. This form of control group may be selected when designing or selecting standardised tasks, or preferably Structured tasks, to a degree of difficulty which extend the measurement range of cognitive ability beyond the norm.

Data is collected during performance of the standardised task for each member of the control cohort, in order to obtain a range or ranges of normalised task completion times and blink characteristics during performance of the chosen task.

The term “control range” as used herein refers to limits of a parameter set to characterise a group of control subjects' for a standardised task, the parameters including task completion time and blink characteristics or derivatives thereof, and non-linear ranges of patterns of blink characteristics, such as variations in patterns of blinks throughout a particular phase (spatial patterns), as well as linear variation in task completion time or blink parameter characteristics for the task, such as a minimum and maximum value for task completion time T. Hence, the term “control range” can refer, for example, to a particular variation within patterns of blink clusters (ie a control blink cluster pattern), which may be best represented by joint distributions over two or more parameters.

The examples and figures describe control ranges and their application in the present invention.

Data from the standardised task can be analysed using a number of different techniques, in order to obtain the above-mentioned control range or ranges. Persons skilled in the art will appreciate that a wide range of suitable, alternative techniques exist for computational analysis of the above-discussed control data, in order to derive control values and/or ranges for a cohort of control subjects, for use in the methods of the present invention.

For example, the control subjects can be compared according to one or more blink characteristics during one or more chosen phases within the standardised task. In a particularly preferred form, temporal occurrence of eyeblinks is measured throughout the task, and a particular phase of the task is chosen for analysis by showing the size of the gaps (G) between successive blinks during that phase of the task. It is also preferable to display a second measure representing blink power (B), which is derived from one or more of eyeblink amplitude and eyeblink duration. The B value can be used to introduce weighting to the value of each blink (rather than unified weighting for each blink).

Various weightings can be used for each blink, for example, by assigning to each B value:
(i) unit weighting (w=1)
(ii) weight by relative a values (w=a)
(iii) weight by relative d values (w=d)
(iv) weight by a×d (w=a×d)
   or the B value can be weighted further by size of gap G such as:
(v) a×d×G or
(vi) a×d/G

A two-dimensional representation of the common phase data of a subject is then possible, by plotting or tracking temporal eyeblink amplitude (gap between blinks or G) against time for the common phase of the task. It is also preferable to plot or track a characteristic of the eyeblinks themselves throughout this phase, and in particular, the eyeblink power for each blink.

In a refinement of the methods for analysis of individual blinks, we may also apply a smoothing function to the incidence of each blink so that nearby blinks which occur naturally as discrete events may be aggregated into a smoothed “density function” (D) in which graphical peaks represent the occurrence of clusters of blinks. Eyeblink density (referred to herein as a D value) may be obtained by applying a smoothing function F such as a normal Gaussian function to the point of incidence of each blink whose standard deviation parameter (bw=SD) is greater than the minimum refractory period between individual blinks for the subject individually or for the control group collectively. The individual F values, and so the aggregated D value, can be unweighted or weighted according to eyeblink amplitude, eyeblink duration, G value, or any derivative thereof.

Clusters of blinks identified within the passage of a Structured task are particularly useful in aligning subjects by subtask completion. The counting of peaks in the smoothed density function D provides a measure of the number of clusters representing completion of subtasks executed by the subject in the completion of the task.

Computational analysis of the blink pattern can be performed using a variety of methods. For example, it is possible to calculate “intensive” parameters by smoothing the blink data using a Gaussian kernel Fx (ie a standard mathematical methodology used to weight variable functions), with any suitable bw value as its standard deviation as discussed above, but computed for a specific period of the task. Preferably this will comprise the common completion phase of the task, defined in duration as the period to completion of the said task by the subject in the control group taking the least time to complete the task.

This weighting function (w) is used to weight each blink in association with a smoothing function to smear the blink signal artificially so that blink clusters may be conveniently aggregated. With a bandwidth (bw) within a range of approximately 1.5-3 seconds for each blink, we may conveniently cluster individual blinks to show the peaks in the density function for blinks which themselves never overlap, as each blink is discrete. This allows the assessment of periods of blink clustering as peaks on a two-dimensional display and the automated counting of associated intensive characteristics of blinking in the common phase of the task, such as the number of peaks (BP) occurring as clusters in the intensive analysis, the area of those peaks corresponding to the number of blinks in the cluster (BC), and the (more random) background blink rate (BB) independent of clusters that may occur for some subjects.

As used herein, the term “intensive parameter” refers to a derived value from one subject, or a range from a group of subjects, that is calculated according to type of blink and the manner in which blinks are clustered during a task, such that intensive parameters are indicative of how a subject completes the common phase of a standardised task.

By selecting a common phase of the standardised task for the intensive and smoothed analysis, it is possible to complement the analysis for each subject and go beyond the conventional “extensive” and discrete blinking parameters for the task, such as the task completion time T, the total number of blinks N for the task, and the total number of attempts A where applicable for a Structured task.

Intensive parameters which may be calculated to characterise each subject during the common task completion phase and extensive parameters for the whole task also measured to characterise each subject. Psychometric tests designed without consideration of the value of detailed blink analysis will normally be designed to discriminate subjects by the extensive parameters of the task (task completion time, number of attempts, number of errors) whereas standardised tasks designed allow for the systematic comparison of how subjects complete a common phase of the selected task by inclusion of the intensive parameters in the analysis.

Suitable intensive parameters include:
percent of blinking time in the common task completion phase (CTCP), a value referred to herein as “I1” (preferably calculated by the percentage of time when blink density is above a baseline density in the last 90 seconds of a task taken by subjects in the control group talking from 90 seconds to 500 seconds to complete the task);
mean peak area of blinks clustered at each concentration release, referred to herein as “I2” (preferably calculated using the integral of height of density in last 90 seconds of the task, divided by the number of peaks in the density function for the task time period, representative of blinks clustered at each concentration release, whereby blink clusters are determined by measuring the area of each cluster peak above the local baseline);
average baseline blink density in last 90 seconds, a value referred to herein as “I3” (preferably derived from the opening function by calculation of the incidence of minimum followed by a maximum in the relative density function within a threshold of 15 seconds or 10% to 20% of the CTCP); and
average blink rate for the task, referred to herein as “I4” (calculated by total number of blinks N over the task on total time T taken for the entire task).

The term “relative density function” D as used herein is the trace of blink incidence which results from the replacement of each blink as it occurs in the temporal incidence of blinks with a smoothing function F, such that physically discrete blink events are represented as overlapping distributions which accumulate into peaks in the density function D whenever blink incidence is close together.

Any one of the above-mentioned intensive parameters may be used to create a control range for control subjects, such that a test subject having an intensive parameter falling outside of that control range has cognitive processing that differs from subjects in the control group.

Intensive parameters are preferably assessed over a single period of time common to all test subjects for a given task (eg time to complete the task, including the point of completion of the task). Intensive parameters reflect derived values such as the background blink rate (BB) during the common period of time in the task; the number of peaks in blink density during the common period (BP); the proportion of time without blinks during the task, and the average blink rate. As individual blinks are discrete events, the blink density must be derived from the individual blink records by applying a smoothing function F to each blink, which is wider than the minimum interblink interval for a given individual or group of individuals, as discussed above. The smoothing function is chosen to emphasise clusters of blinks as they occur during the task.

Typically, blink clusters occur as peaks in the blink density at the point of release of concentration at the end of each progressive attempt during the task. How many blinks aggregate into such a cluster will vary widely between subjects, and a consistent estimate of this parameter is an intensive parameter important in discriminating individuals who share a similar task completion time. These intensive parameters together are chosen to capture information on the proportion of time during the task which is free of blinks and conversely, the number of clusters of blinks occurring in the blink density, and the aggregate size of these clusters (representing the number of blinks in the cluster).

The intensive parameters reflect the intensive clockwork of the brain's subtask processing during the selected common phase of the (Structured) task. As such they are designed to reflect underlying neurological competence (or capability, reflecting the chunking of a task).

The term “extensive parameters” as used herein, refer to values which reflect a subject's aptitude (or ability, or underlying knowledge and experience for such a task). It is possible to calculate several extensive parameters, for the whole of a standardised task. Extensive parameters include:
total length of task (referred to herein as “E1”, which is interchangeable with the term “T”);
total number of blinks (referred to herein as “E2”, which is interchangeable with the term “N”);
number of clusters during entire task time (peaks in blink density) (referred to herein as “E3”); and
indirect measure of number of clusters/attempts at stages in task, calculated by periods of relative rarity of blink occurrences (referred to herein as “E4”), which may be calculated using a formula of attempts (A)=T̂2/sum(gap̂2).

Any one of these extensive parameters may be used to create a control range for control subjects, such that a test subject having an extensive parameter falling outside of that control range has a cognitive aptitude that differs from subjects in the control group.

As already discussed, the control group may represent subjects having established/diagnosed impairments in cognitive processing, thus a test subject who has an extensive or intensive parameter falling outside of the control range does not necessarily have an impaired cognitive function. Rather, the test subject's cognitive function is said to be altered relative to that of the control group.

As used herein, the term “altered cognitive function” refers to cognitive function that is either enhanced or impaired relative to a control range. The use of a control range for more than one parameter may be necessary to position a test subject relative to a control group.

Thus, the methods of the present invention encompass the use of any one or more of these intensive and/or extensive parameters for comparing subjects within a control group, and/or analysing the cognitive aptitude of a test subject relative to a control group, to ascertain whether the test subject's cognitive function is altered relative to that of the control group.

In another form of inter-subject comparison, it is possible to either create a control range using a cohort of control subjects, and/or analyse the cognitive processing of a test subject relative to the control range, by sorting performance in a standardised task according to time to complete the task in the control subjects, and comparing one or more measures of eyeblink function during performance of a particular phase (eg the completion phase) of the task with that of the control subject.

Alternatively or additionally, subjects can be analysed according to task completion times, and in particular, by comparing subjects who have a similar overall task completion time.

Once a control range or ranges have been established, a test subject can be compared with the control group once the test subject has completed the standardised test, and comparing the test subject's performance (ie time to complete the task, blink characteristics, and any derivatives thereof), with those of the control group.

In a particularly preferred form, the control subjects are analysed by aligning the completion phases of the standardised task, for calculation of any one or more of the intensive parameters referred to above. The subjects are then sorted from lowest to longest completion times for the standardised task, and the total number of blinks (N) per subject also displayed in this sorted completion time graph.

Values can be assigned to rank subjects within the control range, such as values within a range of 1 to 3, with 1 corresponding to eyeblinking of high structure (relatively sparse blinking for a similar time to complete the task compared to other subjects), and 3 corresponding to eyeblinking of low structure (relatively dense blinking for a similar time to complete the task compared to other subjects). Subjects who share a similar time to complete the task, and show intermediate number of blinks during the task, are classified as intermediate structured eyeblinking and assigned the value 2 for blink structure.

Linear regression can then be used to find a linear combination of the above-discussed parameters, I1 to I4 and E1 to E4, that best represents relative structure of blinking within the control group. This analysis permits assignment of an “objective Structure index” for each subject, as a single intensive measure of the subjects performance, which may be contrasted with one or more extensive measures from the original rankings (ie subject's time to complete the task). This objective structure index value may be plotted graphically as against a second coordinate for each subject within a control group (eg the time to completion value). Preferably one intensive measure and one extensive measure will be chosen to best discriminate all the subjects in the control group.

Since the objective structure index represents an intensive measure of a blinking pattern, the choice of the second coordinate as an extensive variable (eg task completion time) allows subjects within a grouping to be represented in a graphical spread or map, which reflects processing characteristics or capability (through the objective Structure index) and individual task aptitude (through the time to complete the task for each subject).

### Devices for Performing the Methods of the Invention

Persons skilled in the art will readily recognise that a wide variety of suitable devices exist for gathering the blink and time information necessary for performance of the methods of the invention. Suitable devices include those that collect eyeblink information in the time domain, rather than the frequency domain, and as such, the required eyeblink information can be derived from standard EEG devices by the same mathematical procedures used to characterise eyeblink artefacts within EEG data.

Preferably, eyeblink information is directly measured using the EOG (electrooculargram) voltage of eyeblink parameter data. Use of such direct measurement simplifies data collection such that measurement of a subject's eyeblink pattern during task performance can be taken in a broader range of settings than is possible for EEG measurements.

Accordingly, the most suitable devices for the methods of the invention collect data from time of task initiation and temporal occurrence of eyeblinks. Preferably, the devices also measure parameters for each blink, including eyeblink duration and amplitude (depth of blink, or perclos). The device will be required to measure parameters that are highly informative of the actual timecourse of cognitive processing in relation to specific aspects of the standardised task.

The minimum requirement for a device of the invention is thus a means of physical detection in a subject, of at least one independent parameter of eyeblink characteristics during a standardised task, this characteristic being temporal occurrence of the eyeblink.

That is, for each blink, the first of these parameters identifies the time, t, at which the blink occurs.

Preferably, detection of a second parameter is also performed, which characterises the blink in terms of its size, depth, amplitude and/or duration.

In another form, a device according to the invention comprises a means of displaying the temporal occurrence of the eyeblinks of a subject during the task. Such a display device may also display the above-discussed, second parameter of eyeblink size, depth, amplitude or duration.

Persons skilled in the art will appreciate that numerous variations and modifications will become apparent. All such variations and modifications which become apparent to persons skilled in the art, should be considered to fall within the spirit and scope that the invention broadly appearing before described.

In order that the present invention may be better understood and put into practice, certain embodiments of the invention will now be described by way of the following, non-limiting Examples.

### EXAMPLES

### Example 1

### Materials and Methods

A cohort of control subjects was recruited, consisting of subjects without diagnosed impairments in cognitive processing (eg learning difficulties, ADD etc). A second group, comprising subjects diagnosed with ADHD, consisting of both drug-treated and non-treated subjects, were also recruited for testing.

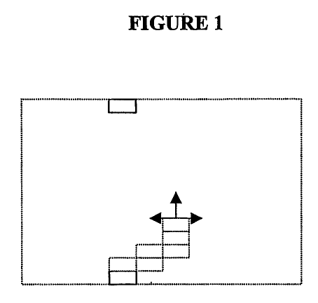

All subjects were required to undergo a maze navigation test as a standardised task. This particular task was an iterative task requiring subjects to navigate the maze using directional instructions of a computer (up, left, right), in order to uncover a hidden track through the maze, as schematically illustrated in FIG. 1.

Completion of the maze without error requires memory and concentration and a coherent sequence of navigation choices without error. Accordingly, the final phase of the maze navigation task was expected to be as common to all subjects as possible, such that the conclusion of the task was expected to be where maximum similarity of cognitive processing demands would be seen. That is, the subjects would already have learned, through earlier phases of the task, how to successfully navigate through the final stages of the maze.

EEG measurements were used to collect the following values for each blink during the task:
t, time since task commencement;
a, blink depth or amplitude (eg as percentage of iris covered) of full blink depth closure (“perclos”); and
d, duration of blink.

EEG measurements are designed to reveal the relative power spectrum of certain frequency bands of neuronal activity. These involve multiple electrodes placed over the scalp during a task. The characteristic EOG signal of an eyeblink was obtainable from the frontal electrodes and its distortion of the power spectrum calculations, particularly near the frontal brain areas near the eyes, (ie that portion of the EEG signal conventionally treated as an artefact). However, it is possible to obtain the eyeblink record directly from the electrode trace, or via a defined, mathematical artefact removal process.

### Results

Wide variations were seen in blink patterns during the passages of the task. However, individual comparisons revealed an underlying similarity in blinking for all subjects, including:
tendency to blink at successful perception
tendency to blink at commencement of action
tendency to withhold blinking during concentrated attention
tendency to release one or more (flutter) of blinks at the end of concentration period

Furthermore, miniblinks were seen to occur that marked internal processing phases of the task.

Since age, prior education experience and skill influence task completion times, blink patterns were investigated for individual differences where (a) subjects took a similar time to complete a task, or (b) comparing the same completion phase of the task. The maze navigation task required ˜90-500 seconds for the group of subjects to complete. This was investigated more intensively by looking at the last common 90 seconds of the task and by comparing subjects with similar task completion times.

### Example 2

### Display of Detailed Blink Data

### Ordering by CTCP Phase

For each subject tested according to the protocol outlined in Example 1, the pattern of blinks was displayed as an ordered set of icons for each blink occurring during the task. The interval between blinks varied widely during the task, therefore the gap (G) between blinks was plotted on the vertical axis of a 2D display at the point in time at which the blink occurs (t).

A control range of G values which is local to a subtask can be obtained from the blink data for different phases of the task if the task is structured, and therefore it is possible to systematically identify periods of relative absence of blinks from periods of relative high incidence of blinks, (denoted herein as clusters). The relative absence of blinks represents the background blink rate, and clusters represent periods of high blink occurrence. The maximum information from the blink intervals comes from comparing the local variation in G values from one stage to another of the task. Therefore, it is the range of the variation in blink rate during a task that provides a more precise indicator of processing behaviour than is possible by averaging all variation into a single number.

Hence, variation in control ranges of G values for the subjects without a cognitive disorder, throughout a selected phase or intra phase stage of the task, are obtained by comparing variation in patterns of the inter blink interval (G) for the control group. This control G range can then be compared with a subject diagnosed with a cognitive disorder at a similar phase/intra-phase stage of the task.

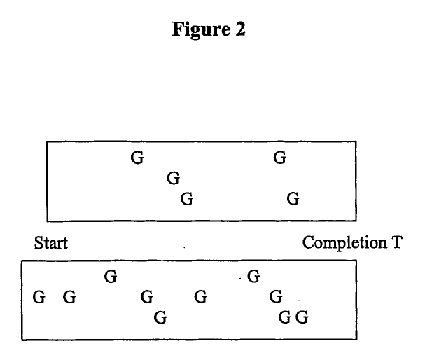

For the CTCP phase, a comparison of the displays was performed by aligning the CTCP phase of the task for direct comparison (FIG. 2).

For the CTCP phase, comparison of intensive measures of blink patterns was also performed, including:
(i) I1=percent of blinking time in the CTCP, when blink density is above a baseline blink density (I1);
(ii) I2=R, the mean cluster size per cluster (integral of blink density divided by number of peaks in last CTCP, representative of blinks clustered at each concentration release, whereby blink clusters are determined by measuring the area of each cluster peak above the local baseline) (I2);
(iii) I3=BB, average baseline blink density in last CTCP (derived from the opening function by calculation of the incidence of minimum followed by a maximum in the relative density function within a threshold of 15 seconds or 10%-20% of the CTCP) (I3);
(iv) I4=N/T overall task blink rate

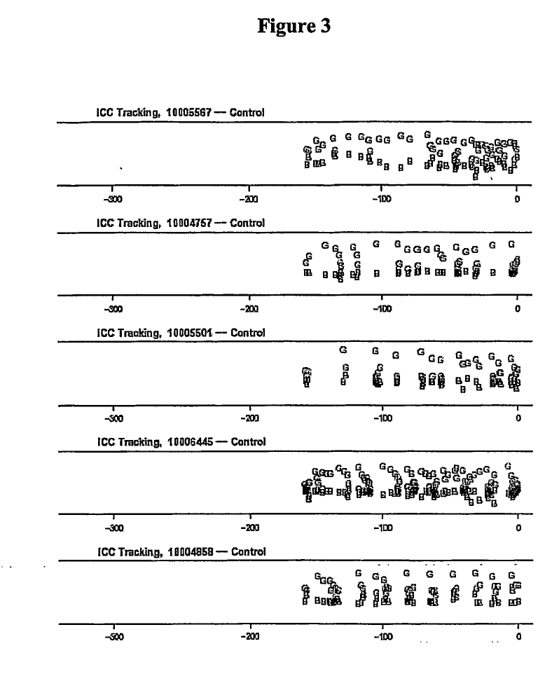

For each blink a second icon was displayed, which indicated blink power (B), estimated as (i) amplitude of blink (a), (ii) duration of blink (d), or (iii) some function of a and d, such as the product: a×d (FIG. 3).

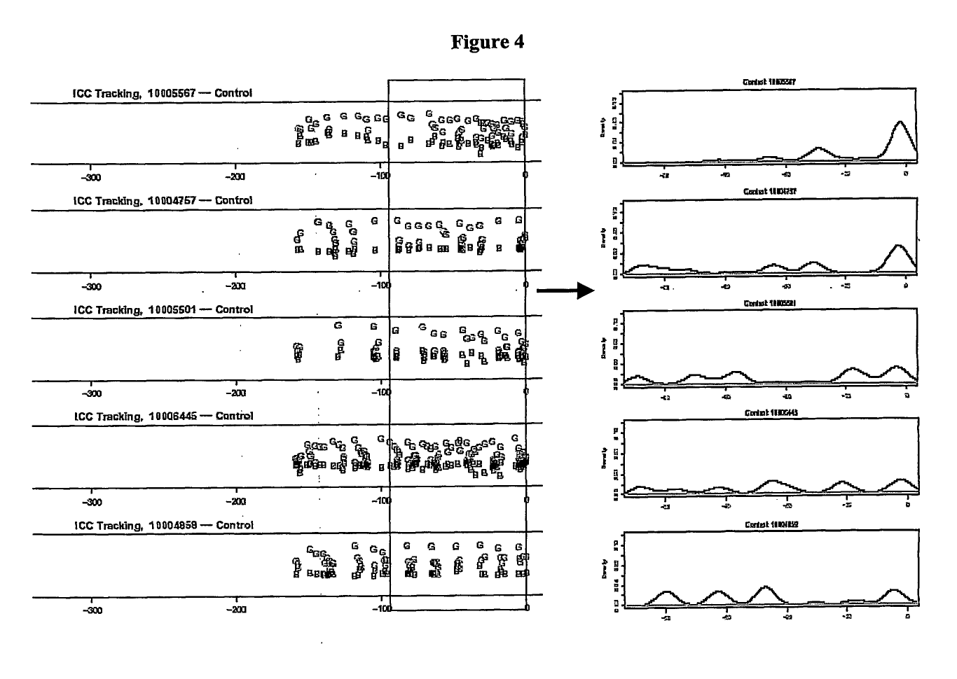

Smoothing of the blink data displayed in FIG. 3 into blink density peaks, which show the clustering of peaks during a selected phase of the task was also performed for the CTCP phase (the last 90 seconds of the task) (FIG. 4).

The B parameter was used to introduce weighting to the assessment of value of each blink. This innovation is in contrast to the default assignments of the same weight (unit weight or count) for each and every blink. On the blink rate (N/T) calculation, each blink was rated the same (unit value) by contrast.

Various weightings were employed for each blink, by assigning to each:
(i) unit weighting (w=1)
(ii) weight by relative a values (w=a)
(iii) weight by relative d values (w=d)
(iv) weight by a×d (w=a×d)

Or weight further by size of gap G such as:
(v) a×d×G or
(vi) a×d/G

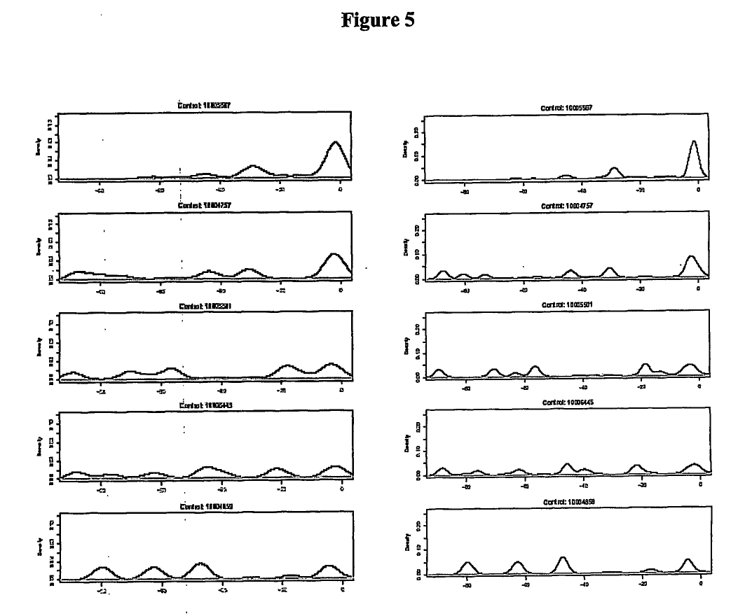

### (FIG. 5).

This weighting function (w) was used to weight each blink in association with a smoothing function to smear the blink signal artificially so that blink clusters may be conveniently aggregated. With a bandwidth (bw) of 1.5-3 seconds for each blink, the peak for blinks which themselves never overlap (each blink is discrete) it was possible to assess periods of blink clustering as peaks on the 2D display. Peaks tended to occur at the conclusion of concentration attention phases, as shown in FIGS. 4 and 5.

Another useful comparative value for differentiating non-ADHD (control) and ADHD subjects was found to be the clustering of peaks during particular time points of the task, and in particular, the CTCP phase. Peak cluster number and/or density at a particular phase point for the control subjects could be used to obtain a range of control values, against which ADHD subjects could be compared.

The background blink rate was estimated through this phase by the opening function (BB).

The number of blinks per cluster was estimated by the peak area above the background blink rate, BB.

### Example 3

### Ordering by Time to Complete the Task

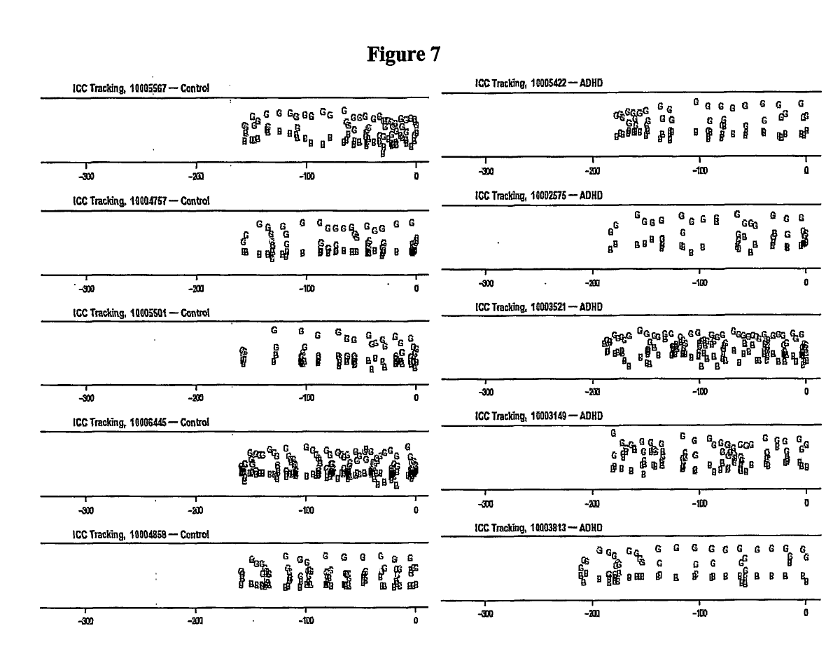

Data obtained according to the protocol outlined in Example 1 was analysed by comparing time to complete the maze navigation task. Subject's blink patterns were aligned by adjusting patterns to the common completion phase of the task, then subjects were sorted from lowest to longest completion time (FIG. 7).

It was observed that blink numbers correlated in general with task completion times, but also wide individual variations were observed in blinks across subjects who shared similar completion times.

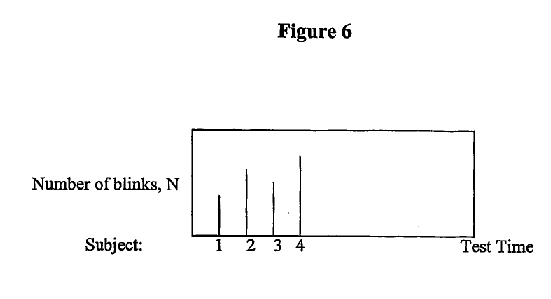

By comparing each subject with nearest neighbours, blinking patterns were identified, including relatively dense blinking (eg N2>N1, N2>N3) and relatively sparse blinking (eg N3<N2, N3<N4) (FIG. 6).

It was possible to obtain a range of blink numbers N for the control group (non-ADH)) subjects, for a given time to complete the task. When N values from the ADHD subject having similar or identical times to complete the task as members of the control group were compared with the control range of N values, a deviation from the control range could be observed for a number of ADHD subjects.

Objective quantification of the characteristics of relatively sparse blinking (structured) or relatively dense blinking (struggling) for a similar task completion time, was also performed using the following method:
i) disordered blinking by combing through similar task completion times to identify those subjects with relatively large blink count d1, d2, d3 . . . a set of relatively disordered subjects (ranked as 3).
ii) structured blinking by combing through similar task completion times to identify those subjects with relatively small blink count o1, o2, o3 . . . a set of relatively ordered subjects (ranked as 1).

Subjects lying near the trend line of Ni (number of blinks for subject i) vs Ti (time taken for subject i) are characterised as intermediate (ranked as 2)

These rankings were correlated by linear regression with both intensive and extensive variables calculated E1-E4, I1-I4 and so objectively characterised and transformed into a linearised measure, the objective Structure Index (SI).

Thus it was possible to distinguish T (extensive task indicator) from order/disorder (relative intensive indicator of the Structure Index) for each subject and map all subjects in these two dimensions.

These two variables, one intensive and one extensive, become the important for discriminating subgroups within each test group.

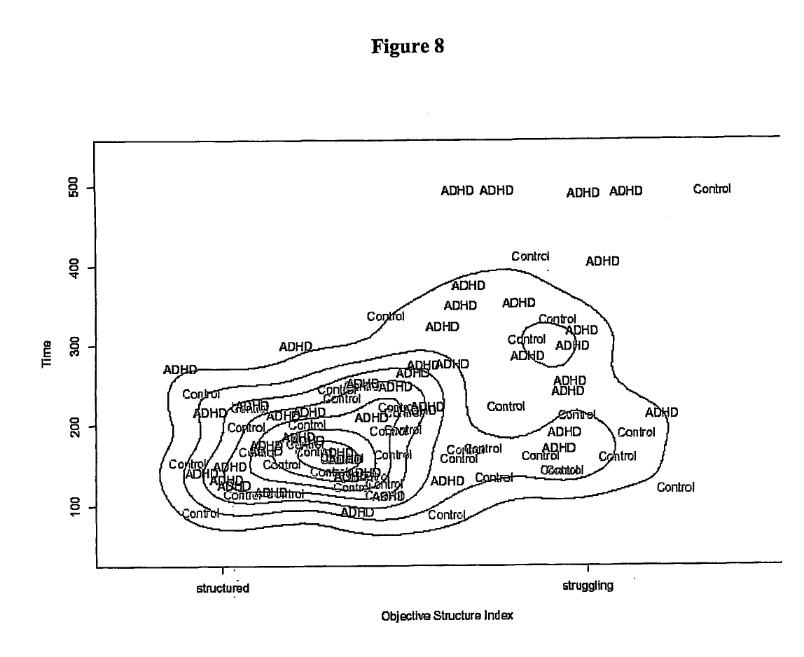

Subtypes reflecting clusters of similar blinking types were found to exist when a sufficiently diverse group was aligned by (a) task completion times and (b) order/disorder index (FIG. 8).

Clusters of subjects identified in various locations can be interpreted as showing consistent subtypes of blink patterns, though display of at least
one extensive variable (eg task completion time) against one intensive variable (eg order/disorder index).

For example subjects with more structured blinking and shorter completion times may be representative of normal blinking, whereas subjects showing bother longer T and more disordered blinking were more characteristic of attention deficit hyperactivity disorder (ADHD) syndrome and subjects having short/usual T but disordered blinking may show dyslexia syndrome (primary comprehension or visual perception difficulties). Treated ADHD subjects show normal profiles typical of Controls.

### Discussion

Maps of the kind shown in FIG. 8 may be used to characterise patient subpopulations. The utility of these maps becomes clear when the ambiguous and non-specific nature of much neurological diagnoses of diffuse syndromes is understood. Thus the ability to objectively characterise both extensive and intensive cognitive processing variables for a given cohort of patents provides the basis for either a:
(i) clear diagnosis of a potential disorder,
(ii) a likely confirmation of a previously suspected disorder, or
(iii) a means of discriminating objective subtypes of the given syndrome.

Persons familiar with neurological diagnosis, and with learning difficulties such as ADHD or Dyslexia, will be aware of the difficulty in precisely characterising subjects:
(a) based on interview/observation; and
(b) use of psychometric test' batteries, which typically show wide variation from age(E)/skill(I)/education(E)/intelligence(I) variables.

1. A method for creating a control range suitable for detecting cognitive processing of at least one test subject using a standardised task, the method comprising:
measurement of time to complete the standardised task in a group of control subjects; and
measurement of temporal eyeblink occurrence during performance of the task by the control subjects; and
calculation of a control range for the control subjects, the control range being calculated from at least the temporal eyeblink occurrence during a common phase within the standardised task for the control subjects;
wherein deviation from the control range indicates the test subject has altered cognitive processing relative to the control subjects.

2. The method according to claim 1, wherein the temporal eyeblink occurrence is used to derive the gap or time elapsed between blinks over a plurality of adjacent blink events.

3. The method according to claim 1, wherein the standardised task is a structured task, where the common phase is selected from:
a first orientation phase, occurring at commencement of the task;
a second or intermediate phase showing the test subject's task progress; and
a common task completion period (CTCP) of the task.

4. The method according to claim 3, wherein the structured task is chosen to emphasise the control subject's mental processing during the orientation phase and/or intermediate phase of the task by incorporation of the orientation phase and/or intermediate phase into later phases of the task.

5. The method according to claim 3, wherein the common phase of the standardised task is the completion phase of the task.

6. The method according to claim 1, wherein measurement of at least one of eyeblink characteristic of eyeblink power B is also performed, the eyeblink characteristic being selected from eyeblink duration and eyeblink amplitude, the eyeblink characteristics occurring during performance of the standardised or structured task.

7. The method according to claim 6, wherein B is a mathematical derivative of eyeblink amplitude and eyeblink duration.

8. The method according to claim 6, wherein B is a mathematical derivative of eyeblink duration, eyeblink amplitude and temporal gap between eyeblink occurrence.

9. The method according to claim 2, wherein the common phase of control subject's performance in the common phase of the task is analysed by plotting or tracking quantitative gap time elapsed between the plurality of blink events against time of the task.

10. The method according to claim 9, wherein measurement of at least one eyeblink characteristic of eyeblink power B is also plotted or tracked for each blink against time of at least the common task, wherein B is selected from eyeblink duration, eyeblink amplitude, temporal gap between eyeblink occurrence or any derivative thereof.

11. The method according to claim 10, wherein individual blinks are smoothed and weighted by clustering into peaks occurring over the common phase within the task.

12. The method according to claim 11, wherein cognitive processing of a first control subject is compared to the cognitive processing of a second control subject, both subjects having a similar task completion time, by comparison of clusters of blinks occurring during the standardised task, to create a control range for clusters of blinks during a common phase of the task for a particular time taken to complete the task.

13. The method according to claim 11, wherein a smoothing function F is used to compare blink clusters on a two-dimensional scale of blink density D against time for the common phase of the task, and wherein number of blinks per cluster is estimated by a peak area above an opening function of a background blink rate (BB), with a converse measure being a proportion of time that blink density does not exceed a baseline of the background blink rate of the blink density.

14. The method according to claim 13, wherein individual blinks are weighted by assigning a weighting using variables selected from unit weighting, absolute amplitude value (a), absolute duration value (d), absolute gap value (G) or a weighting derivative thereof.

15. The method according to claim 14, wherein the weighting derivative is a×d.

16. The method according to claim 14, wherein the weighting derivative is a×d×G.

17. The method according to claim 14, wherein the weighting derivative is a×d/G.

18. The method according to claim 3, which further includes interpretation of the control subjects' performances in the structured task using additional parameters for each control subject, the additional parameters including at least one intensive parameter calculated from the common phase of the task, and at least one extensive parameter calculated from blink patterns over the entire task, the intensive parameter being selected from the group comprising:
percent of blinking time in the CTCP, when blink density is above a baseline blink density (I1);
mean cluster size per cluster (integral of blink density divided by number of peaks in last CTCP, representative of blinks clustered at each concentration release, whereby blink clusters are determined by measuring the area of each cluster peak above the local baseline) (I2);
average baseline blink density in last CTCP (derived from the opening function by calculation of the incidence of minimum followed by a maximum in the relative density function within a threshold of 15 seconds or 10%-20% of the CTCP) (I3); and
average blink rate for the Task (calculated by total number of blinks over the Task on total time taken for the entire Task) (I4),
wherein I1-I3 are derived using a smoothed blink density function F for each blink, and wherein the extensive parameter is selected from the group comprising:
total duration of Task (E1 or T);
total number of blinks (E2 or N);
number of clusters during entire Task time (peaks in blink density) (E3); and
indirect measure of number of clusters/attempts at stages in task (E4), using a formula of attempts (A)=T̂2/sum(gap̂2);
wherein variation in one or more intensive and one or more extensive parameters calculated for the control subjects provides data for the control range for the one or more intensive or extensive parameters.

19. The method according to claim 18, wherein the smoothing function F is a normal Gaussian function.

20. A method according to claim 1, wherein the control group consists of subjects with normal cognitive function.

21. A method according to claim 1, wherein the control group consists of subjects with compromised cognitive function.

22. A method according to claim 1, wherein the control group consists of subjects with enhanced cognitive function.

23. A method according to claim 21, wherein the compromised cognitive function is associated with a disorder selected from the group comprising ADD, ADHD, dyslexia, dementia, schizophrenia, depression, learning disorders, sleep disorder, stress disorder, personality disorders, borderline personality disorders, or cognitive function impaired or enhanced by alcohol or drug ingestion.

24. A method for detecting cognitive processing of at least one test subject using at least one standardised task, the method comprising:
measurement of the subject's time to complete the standardised task; and
measurement of temporal eyeblink occurrence; and
comparison of the temporal eyeblink occurrence or a value derived therefrom, during a common phase within the standardised task, with a control range, the control range calculated from at least the temporal eyeblink occurrence during the common phase within the standardised task for a control group;
wherein deviation from the control range indicates the test subject has altered cognitive processing relative to the control group.

25. The method according to claim 24, wherein the control range is calculated according to the method of claim 1.

26. The method according to claim 25, wherein the test subject's cognitive processing is compared with a control range of one or more intensive and a control range of one or more extensive values.

27. The method according to claim 26, wherein the intensive value is I4, and the extensive value is E3.

28. The method according to claim 24, wherein the test subject's cognitive processing is tested to detect a cognitive disorder selected from the group consisting of ADD, ADHD, dyslexia, dementia, schizophrenia, depression, learning disorders, post-traumatic stress disorder, sleep disorder, personality disorders, borderline personality disorders, or cognitive function impaired or enhanced by alcohol or drug ingestion.

29. A method for creating a control range for detecting cognitive processing of at least one test subject using a standardised task, the method comprising:
measurement of time to complete the standardised task in members of a group of control subjects and measurement of temporal eyeblink occurrence and total eyeblink number N, for the control subjects; and
sorting control subjects from lowest to longest time to complete the task and analysing each control subject having a similar task completion time with respect to their N value;
creation of at least one control range of N values according to the time to complete the task,
wherein deviation from the control range indicates a test subject having altered cognitive processing relative to the control subjects having a similar task completion time.

30. The method according to claim 29, wherein the standardised task is a structured task, where the common phase is selected from:
a first orientation phase, occurring at commencement of the task;
a second or intermediate phase showing the test subject's task progress; and
a common task completion period (CTCP) of the task.

31. The method according to claim 30, wherein the structured task is chosen to emphasise the test subject's mental processing during the orientation phase and/or intermediate phase of the task by incorporation of the orientation phase and/or intermediate phase into later phases of the task.

32. The method according to claim 30, wherein the common phase of the task is the common task completion period (CTCP) of the task.

33. The method according to claim 32, wherein the control subjects' times to complete the task are firstly sorted by adjusting blink patterns to align the CTCP of the task, prior to comparing of control subjects according to their N values during the CTCP of the task.

34. The method according to claim 29, wherein measurement of at least one of eyeblink characteristic of eyeblink power B is also performed, the eyeblink characteristic selected from eyeblink duration and eyeblink amplitude, wherein the eyeblink characteristic occurs during performance of the standardised task.

35. The method according to claim 29, which further includes interpretation of the control subjects' performances in the standardised task by linear regression using additional parameters for each control subject, the additional parameters including at least one intensive parameter calculated from the common phase of the task, and at least one extensive parameter calculated from blink patterns over the entire task, the intensive parameter being selected from the group comprising:
percent of blinking time in last or common task completion period (CTCP), when blink density is above a baseline blink density (I1);
mean cluster size (integral of height of density divided by number of peaks in last CTCP, representative of blinks clustered at each concentration release, whereby blink clusters are determined by measuring the area of each cluster peak above the local baseline) (I2);
average baseline blink density in last CTCP (derived from the opening function by calculation of the incidence of minimum followed by a maximum in the relative density function within a threshold of 15 seconds or 10%-20% of the CTCP) (I3); and
average blink rate for the task (calculated by total number of blinks over the task on total time taken for the entire task) (I4),
wherein I1-I3 are derived using a smoothed blink density function F for each blink, and wherein the extensive parameter is selected from the group consisting of:
total duration of task (E1 or T);
total number of blinks (E2 or N);
number of clusters during entire task time (peaks in blink density) (E3); and
indirect measure of number of clusters/attempts at stages in task (E4), using a formula of attempts (A)=T̂2/sum(gap̂2);
further wherein the linear regression is used to establish a linear combination of at least one of I1 to I4 and at least one of E1 to E4 which best represents the structure of the blinking patterns in the control group, wherein variation in one or more intensive or extensive parameters calculated for the control subjects provides data for the control range for the parameter derived by linear regression.

36. The method according to claim 35, wherein the smoothing function F is a normal Gaussian function.

37. The method according to claim 29, wherein structure of blinking patterns for the group is classified or ranked for similar times to complete the task and according to three ranges derived from N values, the ranges being assigned values between 1 and 3, wherein 1 represents highly structured or sparse blinking, and 3 represents struggling eyeblinking with an eyeblink pattern of low structure or dense blinking, and 2 represents an intermediate structure of eyeblinking.

38. The method according to claim 29, wherein the control group consists of subjects with normal cognitive function.

39. The method according to claim 29, wherein the control-group consists of subjects with enhanced cognitive function.

40. A method according to claim 29, wherein the control group consists of subjects with compromised cognitive function.

41. A method according to claim 40, wherein the compromised cognitive function is associated with a disorder selected from the group consisting of ADD, ADHD, dyslexia, dementia, schizophrenia, depression, learning disorders, post-traumatic stress disorder, sleep disorder, personality disorders, borderline personality disorders, or cognitive function impaired or enhanced by alcohol or drug ingestion.

42. A method for detecting cognitive processing of at least one test subject using at least one standardised task, the method comprising:
measurement of time to complete the standardised task and measurement of temporal eyeblink occurrence and total eyeblink number N, for the test subject; and
comparison of the test subject's N value with a control range of N values corresponding the subject's time to complete the task,
wherein deviation from the control range indicates a test subject having altered cognitive processing relative to the control range.

43. The method according to claim 42, wherein the control range is calculated according to the method of claim 29.

44. The method according to claim 42, wherein the test subject's cognitive processing is compared with a control range selected from intensive and extensive values according to claim 35.

45. The method according to claim 44, wherein the intensive value is I4, and the extensive value is E3.

46. The method according to claim 45, wherein the intensive value is derived from I4 and E3.

47. The method according to claim 42, wherein a set of ranking values for each control subject, assigned according to the method of claim 37, is used to calculate an Objective Structure Index by linear regression from a set of intensive and extensive parameters calculated for each control subject, where the resulting linearised Objective Structure Index value is plotted as an intensive variable for each subject against an extensive variable for each control subject, wherein the extensive variable is T or N or some function of T or N, wherein the test subject may be compared with a joint distribution of the intensive and extensive variable for the control group.

48. The method according to claim 42, wherein the test subject's cognitive processing is tested to detect a cognitive disorder selected from the group consisting of ADD, ADHD, dyslexia, dementia, schizophrenia, depression, learning disorders, post-traumatic stress disorder, sleep disorder, personality disorders, borderline personality disorders, or cognitive function impaired or enhanced by alcohol or drug ingestion.

49. A device suitable for recording temporal occurrence of eyeblinks during a time taken for a subject to complete a standardised task, according to claim 1.

50. A device suitable for recording temporal occurrence of eyeblinks during a time taken for a subject to complete a standardised task, and eyeblink characteristics of eyeblink duration and eyeblink amplitude, according to the method of claim 1.

51. A device suitable for displaying temporal occurrence of eyeblinks during a time taken for a subject to complete a standardised task, according to the method of claim 1.

52. A device suitable for displaying temporal occurrence of eyeblinks during a time taken for a subject to complete a standardised task, and eyeblink characteristics of eyeblink duration and eyeblink amplitude, according to the method of claim 1.

53-56. (canceled)

57. A method of computational analysis for analysing eyeblink data, in order to create a control range for cognitive processing and/or detect the cognitive processing of a test subject, according to the method of claim 1.

58. A method of assessing cognitive processing of a subject by analysis of eyeblink pattern in a standardised task.
